(19) United States
(12) Patent Application Publication
(10) Pub. No.: US 2020/0237493 A1
(43) Pub. Date: Jul. 30, 2020

(54) DEVICES, SYSTEMS, AND METHODS FOR TIGHTENING, FASTENING, OR ANCHORING IN TISSUE

(71) Applicant: Boston Scientific Scimed, Inc., Maple Grove, MN (US)

(72) Inventors: Ajay Gupta, Shoreview, MN (US); Cass A. Hanson, St. Paul, MN (US)

(21) Appl. No.: 16/751,798

(22) Filed: Jan. 24, 2020

Related U.S. Application Data
(60) Provisional application No. 62/796,709, filed on Jan. 25, 2019.

(51) Int. Cl. A61F 2/00

(52) U.S. Cl. CPC A61F 2/00; A61F 2220/0016

(57) ABSTRACT

The present disclosure relates generally to the field of medical devices. In particular, the present disclosure relates to medical devices, systems, and methods for tightening, fastening, or anchoring in tissue. In one example, a system for tightening, fastening, or anchoring in tissue, may comprise a spring coil having a distal end, a stressed configuration, and an unstressed configuration; and a restraining tube having a lumen extending along a length of the tube. The spring coil may be removably receivable within the lumen to restrain the spring coil in the stressed configuration. Other embodiments are contemplated.

### PRIORITY

This application claims the benefit of priority under 35 USC § 119 to U.S. Provisional Patent Application Ser. No. 62/796,709, filed Jan. 25, 2019, which is incorporated by reference herein in its entirety and for all purposes.

### FIELD

The present disclosure relates generally to the field of medical devices. In particular, the present disclosure relates to medical devices, systems, and methods for tightening, fastening, or anchoring in tissue.

### BACKGROUND

Medical professionals sometimes face significant technical challenges when delivering a medical device to a body tissue. The device may need to be implanted in proximity to the tissue for an extended period of time. The medical professional may need to ensure that the device is safely secured to the tissue such that the device does not migrate, and the tissue and nearby anatomy is not damaged. Current devices may use sutures that may only be useful for tissue surface devices and may not be long-lasting. Adhesives may also not adhere the device to the tissue for an extended period. Screws may unscrew themselves over time and may cause excessive trauma to patient anatomies.

Helical anchors may typically be deployed and fixated into tissue via rotation. An ongoing difficulty with helical anchors is that a physician may have no reliable way to determine exactly when the helical anchor is fully embedded into tissue at a desired implantation site. Additionally, a helical anchor may dislodge partially or completely from the tissue through natural movement of the patient (e.g., unscrewing), which may result in the anchor migrating into an undesirable position in the patient over time. A physician, not knowing if a helical anchor is fully embedded into tissue, may also over-rotate the helical anchor into the tissue. The result of such over-rotation can be damaging to the tissue and nearby anatomy and may improperly deliver the device.

It is with these considerations in mind that a variety of advantageous medical outcomes may be realized by the medical devices, systems, and methods of the present disclosure.

### SUMMARY

Embodiments of the present disclosure may assist generally with tightening, fastening, or anchoring in tissue reliably. In one aspect, a system for tightening, fastening, or anchoring in tissue may include a spring coil that may have a distal end, a stressed configuration, and an unstressed configuration. A restraining tube may have a lumen extending along a length of the tube. The spring coil may be removably receivable within the lumen to restrain the spring coil in the stressed configuration. A tip with a distal tissue-penetrating portion may be on the distal end of the spring coil. The distal tissue-penetrating portion of the tip may have a sharpened point. A maximum diameter of the tip may substantially match and may abut an outer diameter of the restraining tube. The tip may include a proximal portion that has a proximally decreasing taper, a proximally extending barb, a distally waning frustum, or a proximally extending hook, or a combination thereof. The restraining tube may be disposed over the proximal portion of the tip when the spring coil is in the stressed configuration. A plurality of windings of the spring coil may have a stressed diameter in the stressed configuration and an unstressed diameter in the unstressed configuration. The unstressed diameter may be smaller than the stressed diameter. The restraining tube may be a dissolvable coating. The system may include a delivery catheter having a distal end, wherein a proximal end of the restraining tube may be fixedly attached to the distal end of the catheter. The spring coil may be a helical coil. The restraining tube may be a helical tube. The catheter may be rotatable to deliver the spring coil from within the lumen of the restraining tube. A diameter of the helical tube may be greater than a diameter of the helical coil in the unstressed configuration, or windings of the helical coil may have a pitch that is greater than windings of the helical coil in the unstressed configuration, or both.

In another aspect, a system for tightening, fastening or anchoring in tissue may include a hollow spring coil that may have a lumen extending along a length of the coil and may have an open proximal end, a distal end, a stressed configuration, and an unstressed configuration. A restraining element may be disposed within the lumen. The restraining element may be removably receivable within the lumen to restrain the hollow spring coil in the stressed configuration. A tip with a closed distal tissue-penetrating portion may be on the distal end of the hollow spring coil. The tissue-penetrating portion of the tip may have a sharpened point. A plurality of windings of the hollow spring coil may have a stressed diameter in the stressed configuration and an unstressed diameter in the unstressed configuration. The unstressed diameter may be smaller than the stressed diameter. The restraining element may be a pressurized fluid. A delivery catheter may have a distal end wherein a proximal end of the restraining element may be attached to the distal end of the catheter. A diameter of the restraining element may be greater than a diameter of the hollow spring coil in the unstressed configuration, or windings of the restraining element may have a pitch that is greater than windings of the hollow spring coil in the unstressed configuration, or both.

In another aspect, a method of tightening, fastening or anchoring in tissue may include inserting a catheter including a device into a patient. The device may include a spring that may have a stressed configuration and an unstressed configuration. The device may include a restraint against the spring that may restrain the spring in the stressed configuration. The spring may be deployed from the catheter at least partially into the tissue. The restraint may be withdrawn from the spring such that at least a portion of the spring transitions from the stressed configuration to the unstressed configuration in the tissue to tighten, fasten or anchor in the tissue. The restraint may be a hollow tube about the spring. The restraint may be a coil within the spring. Deploying the spring may include rotating the catheter in a clockwise direction. Withdrawing the restraint may include rotating the catheter in a counter-clockwise direction. Withdrawing the restraint may include fixing a position of the spring relative to the restraint with a push member abutting a proximal end of the spring. The restraint may be attached to a distal end of the catheter. A secondary device may be positioned within the tissue. The spring may be deployed into the tissue about the secondary device. The restraint may be withdrawn such that the spring in the unstressed configuration tightens the tissue about the secondary device.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a spring coil for tightening, fastening, or anchoring in tissue, according to an embodiment of the present disclosure.

FIG. 2 illustrates a spring coil for tightening, fastening, or anchoring in tissue in an unstressed configuration, according to an embodiment of the present disclosure.

FIG. 3 illustrates a restraining tube of a system for tightening, fastening, or anchoring in tissue, according to an embodiment of the present disclosure.

FIG. 4A illustrates a system for tightening, fastening, or anchoring in tissue in a stressed state, according to an embodiment of the present disclosure.

FIG. 4B illustrates a top view of the embodiment of FIG. 4A.

FIG. 5A illustrates a system for tightening, fastening, or anchoring in tissue with a spring coil initially being delivered into the tissue, according to an embodiment of the present disclosure.

FIG. 5B illustrates the system of FIG. 5A with more of the spring coil being delivered.

FIG. 6 illustrates a system for tightening, fastening, or anchoring in tissue including a hollow spring coil and a restraining element, according to an embodiment of the present disclosure.

FIGS. 7A-7D illustrate various tips for a system for tightening, fastening, or anchoring in tissue, according to embodiments of the present disclosure.

FIG. 8 illustrates a system for tightening, fastening, or anchoring in tissue with a delivery catheter, according to an embodiment of the present disclosure.

FIG. 9 illustrates another system for tightening, fastening, or anchoring in tissue with a delivery catheter, according to an embodiment of the present disclosure.

FIG. 10A illustrates a system for tightening, fastening, or anchoring in tissue about a medical device, including a spring coil and restraining tube, according to an embodiment of the present disclosure.

FIG. 10B illustrates the system of FIG. 10A with the restraining tube being removed.

FIG. 10C illustrates the system of FIGS. 10A and 10B with the restraining tube removed.

FIG. 11 illustrates a system for tightening, fastening, or anchoring in tissue with a spring coil about a medical device within two tissue layers.

FIG. 12 illustrates a system for tightening, fastening, or anchoring in tissue fixating a medical device relative to tissue, according to an embodiment of the present disclosure.

FIG. 13A illustrates a system for tightening, fastening, or anchoring in tissue with a J-hook spring coil in a stressed configuration within a restraining tube, according to an embodiment of the present disclosure.

FIG. 13B illustrates the system of FIG. 13A with the end of the J-hook spring coil in an unstressed configuration.

FIG. 14 illustrates a system for tightening, fastening, or anchoring in tissue with an erodible restraining layer, according to an embodiment of the present disclosure.

FIG. 15 illustrates a system for tightening, fastening, or anchoring in tissue with a hollow spring coil containing a pressurized fluid, according to an embodiment of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with reference to body tissue, it should be appreciated that such systems and methods may be used in a variety of anatomies and with a variety of medical devices such as securing devices that may otherwise migrate or need to later be removed, such as pacemaker leads, inferior vena cava filters, vascular plugs, esophageal stents, aortic aneurism grafts, drainage tubes, and the like.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a medical device into a patient, while the term “proximal” refers to the end closest to the medical professional when introducing a medical device into a patient.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

Embodiments of the present disclosure may include devices, systems and methods for tightening, fastening, or anchoring in, tissue and may deliver a stressed coil into tissue to accomplish the same. Delivering a stressed coil into tissue may be performed with direct visual, fluoroscopic, ultrasonic guidance, or the like. The stressed coil may transition in the tissue to an unstressed state that further engages the tissue. The coil may also be used to secure a device to the coil and to the tissue. A coil may be implanted in a patient for an extended period and may be removable.

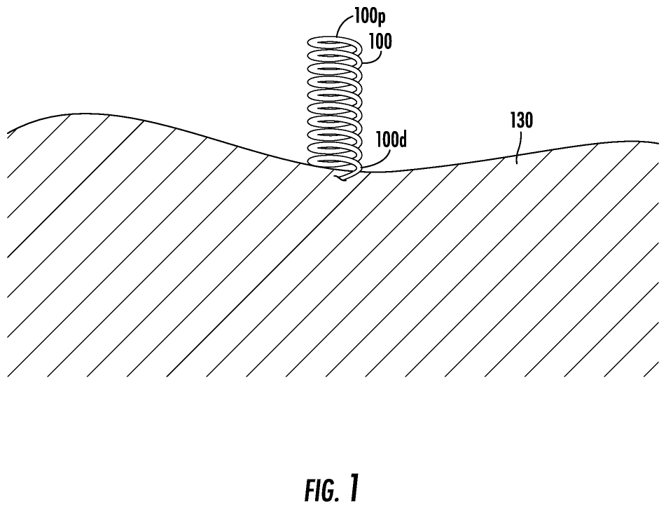

With reference to FIG. 1, an embodiment of a system for tightening, fastening, or anchoring in tissue according to the present disclosure is illustrated, which includes a spring coil 100 anchored into a tissue 130. The spring coil 100 is helical and is in an unstressed configuration and has been delivered into the tissue 130. At least a portion of a distal end 100d is embedded into the tissue 130 such that one or more windings of the spring coil 100 are within the tissue 130. The proximal end 100p of the spring coil 100 is oriented substantially away from the tissue 130 and is unobstructed from access by a medical professional and may be attached to a medical device. The unstressed configuration of the spring coil 100 has the windings of the coil 100 pinching and/or compressing portions of the tissue 130 in which the spring coil is embedded, such that the spring coil 100 is fixed in position relative to the tissue 130.

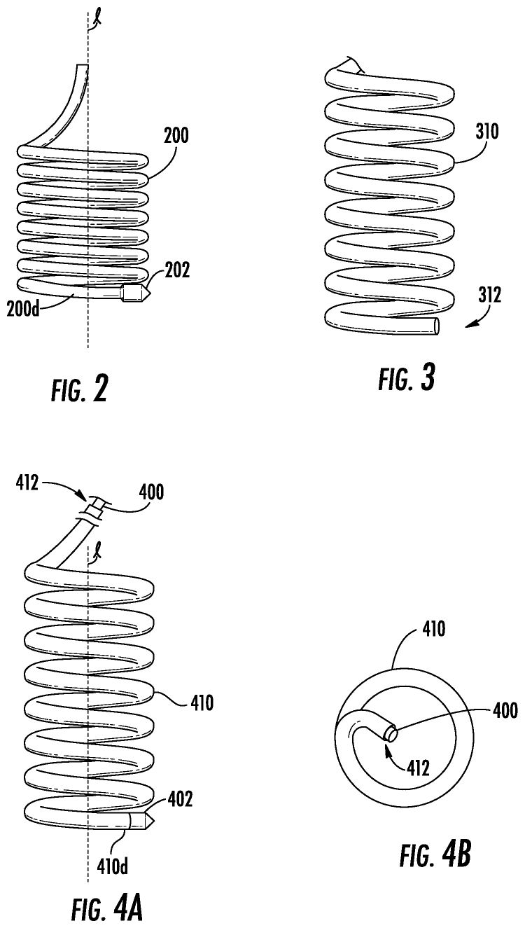

With reference to FIG. 2, an embodiment of a spring coil 200 of a system for tightening, fastening, or anchoring in tissue according to the present disclosure is illustrated. The spring coil 200 is shown in FIG. 2 in an unstressed configuration, where the adjacent windings of the spring coil 200 are closer to each other than in a stressed configuration (e.g., as shown in FIG. 4A, as will be discussed). The spring coil includes a tip 202 with a distal portion having a sharpened point for penetrating tissue a proximal portion that has a radially inward tapering surface that may assist with removing the spring coil 200, and an intermediate portion that transitions from the distal portion to the proximal portion. The distal portion is located on the distal end 200d of the spring coil 200. The spring coil 200 may be transitioned from the unstressed configuration to a stressed configuration by the action of one or more directional forces such as, e.g., tension along the longitudinal axis 1, a restraining force separating the adjacent windings of the coil 200 from each other and preventing their convergence toward each other (i.e., increasing the pitch of the spring coil 400), or the like.

With reference to FIG. 3, an embodiment of a restraining tube 310 of a system for tightening, fastening, or anchoring in tissue according to the present disclosure is illustrated. The restraining tube 310 has a lumen 312 extending along a length of the tube 310. The restraining tube 310 is more rigid than the spring coil 200 of FIG. 2. The rigidity of the restraining tube 310 may be accomplished by having a larger mass than the spring coil 200 and/or by comprising a more rigid material.

With reference to FIGS. 4A and 4B, an embodiment of a system for tightening, fastening, or anchoring in tissue according to the present disclosure is illustrated, which includes a spring coil 400 removably received within a lumen 412 of a restraining tube 410. A diameter of the lumen 412 of the restraining tube 410 substantially matches a diameter of the wire of the spring coil 400 such that the lumen 412 of the restraining tube 410 has a close fit with the spring coil 400 while still being removable from about the spring coil. The restraining tube 410 is shown in FIG. 4A restraining the spring coil 400 in a stressed configuration. The spring coil 400 is elongated along the longitudinal axis 1 in the stressed configuration, and the adjacent windings of the spring coil 400 are farther apart from each other than in the unstressed configuration (e.g., as shown in FIG. 2 and discussed above). When the spring coil 400 is within the lumen 412 of the restraining tube 410, the more rigid restraining tube 410 (compared to the spring coil 400) forces the less rigid spring coil 400 (compared to the restraining tube 410) into the stressed configuration. When the restraining tube 410 begins to be removed from the spring coil 400, any revealed portion of the windings of the spring coil 400 outside of the lumen 412 may transition from the stressed configuration to the unstressed configuration. Although the proximal end of the spring coil 400 is illustrated as bending internally into the helix of the spring coil 400, the proximal end may instead continue in the same helical shape, e.g., to ease removal of the restraining tube 410 from the spring coil 400. In embodiments, the restraining tube 410 may be completely removed from the spring coil 400. In other embodiments, the restraining tube 410 may be retained on a proximal portion of the spring coil 400 after the distal portion of the coil is deployed, e.g., to allow for reconstrainment of the coil in the event of repositioning or removal. The proximal portion of the spring coil 400 may continue its helical shape proximally away from a tip 402 and/or away from a proximal end of the restraining tube 410 and may terminate with the proximal end of the spring coil 400 being wider than the lumen 412 of the restraining tube 410 to aid in keeping the tube from being removed past the proximal end of the coil. The restraining tube 410 may be twisted/rotated away from the tip 402 of the spring coil 400 and remain on the proximal portion of the spring coil, maintaining its helical shape. The restraining tube 410 may be left on the proximal portion of the spring coil 400 temporarily or permanently. The tip 402 of the spring coil 400 includes a distal portion for penetrating tissue. The distal end 410d of the restraining tube 410 substantially abuts the intermediate portion of the tip 402 such that there is a substantially continuous surface from an outer surface of the restraining tube 410 to an outer surface of the intermediate portion of the tip 402; this is because an outer diameter of the tip 402 substantially matches an outer diameter of the restraining tube 410. Together, the system, including the spring coil 400 and the restraining tube 410, may be rotated (e.g., screwed) into a tissue by distally leading with the tip 402. First, the tip 402 leads into the tissue by being pushed by the restraining tube 410, the coil 400, and/or a pusher member (not illustrated), followed by the remainder of the spring coil 400 and the restraining tube 410, which initiates the system embedding into the tissue. The restraining tube 410 may be smoothly embedded within the tissue as the system is rotated generally about the longitudinal axis 1 because of the substantially smooth transition from the tip 402 to the restraining tube 410 during insertion. The sharpened point of the tip 402 creates a path through the tissue during insertion of the system into the tissue and the point widens proximally toward the proximal portion of the tip 402 to accommodate the wider diameter of the restraining tube 410 compared to the point.

In various embodiments, a coil may change shape between configurations. For example, a coil may have a first pitch between windings of the coil in the stressed configuration that is larger than a second pitch between the windings in the unstressed configuration. The transition from the larger pitch to the smaller pitch may cause the coil to pinch the tissue, device, and/or suture between the windings. This pinching may further fixate the coil and any attachments to the tissue. Additionally, a coil may have a first diameter in the stressed configuration that is larger than a second diameter in the unstressed configuration. The transition from the larger diameter to the smaller diameter may cause the coil to squeeze the tissue, device, and/or suture within the coil. This squeezing may further fixate the coil and any attachments to the tissue.

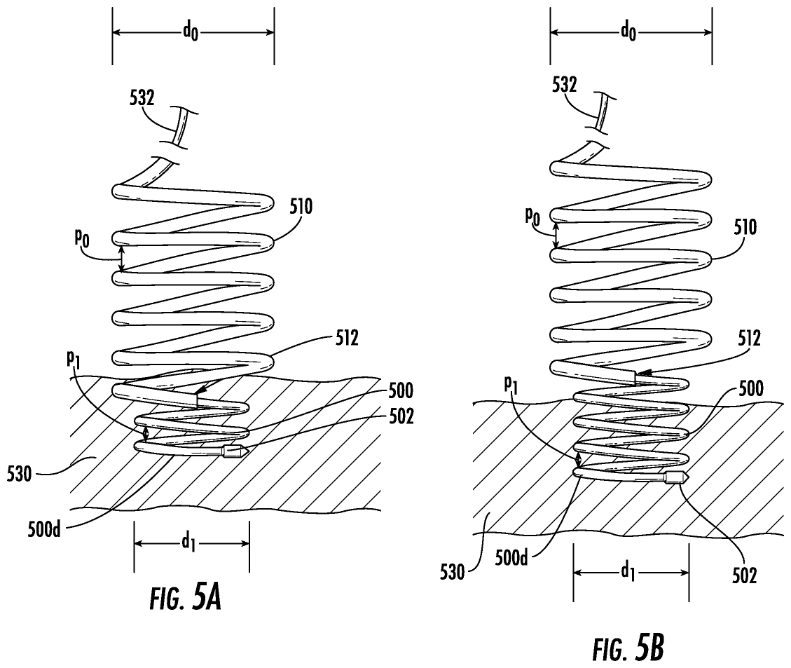

With reference to FIGS. 5A and 5B, an embodiment of a system for tightening, fastening, or anchoring in tissue according to the present disclosure is illustrated, which includes a spring coil 500 removably received within a lumen 512 of a restraining tube 510. In FIG. 5A, the system is shown partially inserted into a tissue 530. This may be done, e.g., by the rotational process discussed above with reference to FIGS. 4A and 4B. The system is inserted into the tissue 530 by leading firstly with a tip 502 of the spring coil 500, which is pushed by the restraining tube 510, the coil 500, and/or a pusher member 532. During insertion, the spring coil 500 is within the lumen 512 of the restraining tube 510. The configuration and greater rigidity of restraining tube 510 compared to the spring coil forces the spring coil 500 into a stressed configuration, such that the windings of the spring coil 500 increase in pitch to a pitch p₀ of the restraining tube 510 and the windings of the spring coil 500 increase in diameter to a larger diameter d₀ of the tube 510 (compared to a diameter of the windings of the coil 500 in an unstressed configuration). After the system is partially inserted into the tissue 530, the restraining tube 510 is moved proximally from the distal end 500d of the spring coil 500. This removal of the restraining tube 510 from the spring coil 500 may be performed by fixating the position of the spring coil 500 with respect to the restraining tube 510, and moving the restraining tube 510 proximally (e.g., by pulling or rotating the restraining tube 510). The position of the spring coil 500 may be fixated with respect to the restraining tube 510 by a pusher member 532 that is positioned to abut up against the proximal end of the spring coil 500. FIG. 5A illustrates the restraining tube 510 initially being removed from the spring coil 500. The distal end 500d of the spring coil 500, including multiple windings of the spring coil 500, is revealed in the tissue 530 (i.e., these windings are outside of the lumen 512). The distal end 500d of the spring coil 500 transitions to the unstressed configuration while the remainder of the spring coil 500 within the restraining tube 510 is in the stressed configuration. The unstressed distal end 500d of the spring coil 500 has a pitch p₁ and a diameter d₁ that are each smaller than the pitch p₀ and diameter d₀ of the tube in which the remainder of the spring coil 500 is constrained in the stressed configuration. This smaller pitch p₁ of the unstressed distal end 500d of the spring coil 500 pinches the tissue 530 between the windings of the distal end 500d. The smaller diameter d₁ of the distal end 500d radially squeezes the tissue 530 within the diameter of the windings. This pinching and squeezing of the tissue 530 fixates the spring coil 500 within the tissue 530 such that the restraining tube 510 can be entirely removed from about the spring coil 500 (e.g., onto the pusher member 532 and/or into a catheter). In FIG. 5B, the restraining tube 510 is shown further removed from the spring coil 500. The revealed portion of the spring coil 500 that is outside of the lumen 512 is in the unstressed configuration, having the smaller pitch p₁ than the remainder of the spring coil 500 that is within the lumen 512. The additional revealed portion of the spring coil 500 having a smaller pitch p₁ further pinches and squeezes the tissue 530 and strengthens the fixation of the spring coil within the tissue 530. The restraining tube 510 may be re-applied to the spring coil 500, transitioning the coil 500 from the unstressed configuration to the restrained configuration. This may allow for the coil 500 and tube 510 to be removed and/or repositioned from/within the tissue 530. Alternatively, a sheath may be applied to the coil 500 to encase the proximal portion of the tip 502 of the coil 500 before retracting the coil 500 from the tissue 530 or repositioning the coil 500 while in the unstressed configuration. The restraining tube 510 may be further removed such that the spring coil 500 is no longer within the lumen 512 and is left alone within the tissue 530 without the restraining tube 510 (e.g., as shown in FIG. 1).

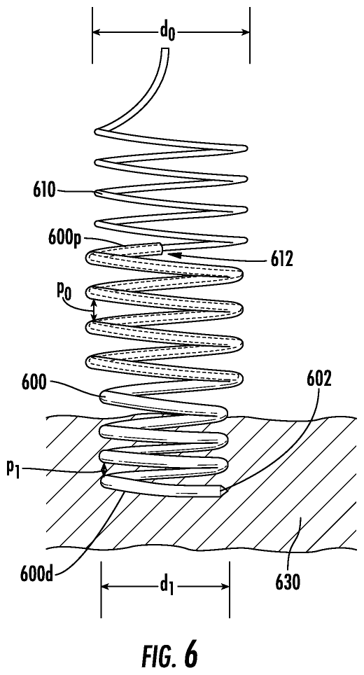

With reference to FIG. 6, an embodiment of a system for tightening, fastening, or anchoring in tissue according to the present disclosure is illustrated, which includes a hollow spring coil 600 having a lumen 612 extending along a length of the coil 600. The coil 600 has an open proximal end 600p and a distal end 600d. A restraining element 610 is disposed within the lumen 612. The restraining element 610 is removably receivable within the lumen 612 and restrains the hollow spring coil 600 in a stressed configuration when the element 610 is within the coil 600. The hollow spring coil 600 includes a closed distal tip 602 that is sharpened for penetrating tissue 630. The system is shown inserted partially into the tissue 630. Insertion may be accomplished, e.g., by rotating the restraining element 610 and/or the hollow spring coil 600 in a counter-clockwise direction such that a distal end of the coil 600 penetrates the tissue 630. The system is inserted into the tissue 630 by leading firstly with the tip 602 of the hollow spring coil 600, which may be pushed by the restraining element 610, the coil 600, and/or a pusher member (not illustrated) about the element 610 and proximal to the coil 600. During insertion, the restraining element 610 is within the lumen 612 of the hollow spring coil 600. The restraining element 610 forces the hollow spring coil 600 into a stressed configuration, wherein the spring coil 600 increases in pitch to a pitch p₀ of the element, and the windings of the spring coil 600 increase to a larger diameter d₀ of the element (compared to the pitch and diameter of the coil 600 in an unstressed configuration). After the system is partially inserted into the tissue 630, the restraining element 610 is moved proximally from the distal end 600d of the spring coil 600. This removal of the restraining element 610 from the spring coil 600 may be performed by fixating the spring coil 600 with respect to the restraining element 610, and moving the restraining element 610 proximally (e.g., by pulling or clockwise rotation of the restraining element 610). The spring coil 600 may be fixated with respect to the restraining element 610 by a hollow pusher member (e.g., as will be discussed with FIG. 8 below) that is positioned to abut the proximal end of the spring coil 600. In FIG. 6, the restraining element 610 is shown initially being removed from the spring coil 600. The lumen 612 at the distal end 600d of the spring coil 600, including multiple windings of the spring coil 600, has been vacated of the restraining element 610 within the tissue 630 (i.e., these windings of the restraining element 610 are not within the portion of the lumen 612 at the distal end 600d of the coil 600). The distal end 600d of the hollow spring coil 600 is in the unstressed configuration, while the remainder of the spring coil 600 having the restraining element 610 still within the lumen 612 is in the stressed configuration. The unstressed distal end 600d of the spring coil 600 has a pitch p₁ and a diameter d₁ that are each smaller than the pitch p₀ and diameter d₀ of the restraining element, in which the remainder of the spring coil 600 is restrained in the stressed configuration. This smaller pitch p₁ of the unstressed distal end 600d of the spring coil 600 pinches the tissue 630 between the windings of the distal end 600d. The smaller diameter d₁ of the distal end 600d radially squeezes the tissue 630 within the windings. This pinching and squeezing of the tissue 630 fixates the spring coil 600 within the tissue 630 such that the restraining element 610 can be removed from within the lumen 612 of the hollow spring coil 600 without removing the spring coil 600 from the tissue 630. The restraining element 610 may be further removed such that it is no longer within the lumen 612 and the hollow spring coil 600 is left alone within the tissue 630 without the restraining element 610.

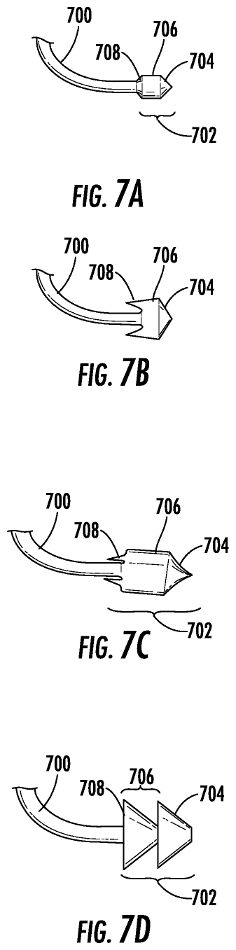

With reference to FIGS. 7A-7D, embodiments of a tip for a system for tightening, fastening, or anchoring in tissue according to the present disclosure are illustrated. These tips may be used interchangeably or in combination with the tips of system embodiments described herein. In FIG. 7A, a distal end of a spring coil 700 includes a tip 702 with a sharpened distal portion 704 for penetrating tissue. The distal portion 704 transitions proximally to an intermediate portion 706 having a substantially straight and substantially cylindrical surface with a diameter that substantially matches the widest cross-section of the distal portion 704 and proximal portion 708. The intermediate portion 706 transitions proximally into a proximal portion 708 that has a radially inward tapering surface (i.e., a frustum that decreases in diameter in a proximal direction toward the coil 700). This proximal portion 708 increases in diameter gradually to the wider intermediate portion 706 (i.e., the proximal portion 708 includes a proximally decreasing taper along the coil 700), which may allow for an easier removal of the spring coil 700 compared to an abrupt widening diameter between the coil 700 and the proximal portion 708 as the coil 700 is proximally removed from a tissue. Although the intermediate portion 706 has an outer diameter that is larger than the spring coil 700, the tapered proximal portion 708 provides a gradual transition from the smaller diameter of the coil 700 to the larger diameter of the intermediate portion 706 for tissue to slide along during removal of the spring coil 700. In FIG. 7B, the spring coil 700 has a tip 702 with a sharpened distal portion 704 for penetrating tissue. The distal portion 704 transitions proximally to an intermediate portion 706 having a substantially radially inward tapering surface (i.e., a frustum that decreases in diameter in a proximal direction). The intermediate portion 706 transitions proximally into a proximal portion 708 that has sharpened barbs configured to engage tissue in a proximal direction (away from the distal portion 704). This proximal portion 708 may provide an additional tissue engagement feature to resist undesirable removal of the spring coil 700 during removal of a restraining tube or restraining element, or undesirable movement of the spring coil 700 after implantation. The intermediate portion 706 may have a variable diameter that decreases in a proximal direction such that a restraining tube may enclose around the proximal portion 708, but not advance to the distal portion 704. In FIG. 7C, a distal end of a spring coil 700 includes a tip 702 with a sharpened distal portion 704 for penetrating tissue. The distal portion 704 transitions proximally to an intermediate portion 706 having a substantially straight and substantially cylindrical surface with a diameter that substantially matches the widest cross-section of the distal portion 704 and proximal portion 708. The intermediate portion 706 transitions proximally into a proximal portion 708 that includes substantially proximal-facing barbs. In FIG. 7D, a distal end of a spring coil 700 includes a tip 702 with a distal portion 704 having a distally waning frustum for penetrating tissue. The distal portion 704 transitions proximally to an intermediate portion 706 having a second redundant distally waning frustum. The intermediate portion 706 transitions proximally into a proximal portion 708 that is a substantially transverse surface (i.e., transverse with respect to a longitudinal axis of the coil 700). This proximal portion 708 may abut with a restraining tube. The proximal portion 708 and the intermediate portion 706 may provide additional resistance to undesirable movement of the spring coil (e.g., in a generally proximal direction). There are no substantially sharp surfaces in this embodiment, which may reduce trauma to the tissue and surrounding anatomy. A restraining tube or a sheath may abut and/or at least partially cover an intermediate portion 706 of a coil 700 having a tip of FIGS. 7A through 7D. The proximal portion 708 of any of these tips may be substantially enclosed by a restraining tube or a sheath such that the features of the proximal portion 708 are not exposed to tissue during insertion, repositioning, or removal of the coil within the tube. In embodiments with an inner spring coil, the intermediate portion of any of these tips may have a diameter that substantially matches a diameter of a restraining tube or a sheath such that when the restraining tube or sheath abuts the intermediate portion, the proximal portion is enclosed within a lumen of the restraining tube. The proximal portion of a tip may only be exposed after removal of the restraining tube, possibly preventing undesirable tissue trauma from resistive features during delivery. The spring coils shown and described in these figures having a distal tip may be a hollow spring coil. A spring coil having a distal tip of any of FIGS. 7A through 7D may be removed from a tissue by a rotational force strong enough to overcome any frictional resistance from a proximal portion of the tip. Alternatively, a tube or sheath may be applied or reapplied about the coil and the proximal portion such that the frictional features of the proximal portion may be substantially encased away from the tissue during removal or repositioning. Various sections of the portions illustrated in these figures may be located along the length of a coil in addition to or instead of the tip.

With reference to FIG. 8, an embodiment of a system for tightening, fastening, or anchoring in tissue according to the present disclosure is illustrated, which includes a spring coil 800 within a restraining tube 810 being directed toward a tissue 830 through the use of a delivery catheter 834, which may be a hypotube. The catheter 834 illustrated in FIG. 8 includes slits that assist with articulation and flexibility of the catheter 834. The spring coil 800 and restraining tube 810 may be inserted into the tissue 830 by rotating the coil 800 and tube 810 into the tissue 830. This rotation may be performed by rotating the catheter 834 or by rotating the coil 800 and the tube 810 independent of the catheter 834. The restraining tube 810 may be attached to the catheter 834 such that the restraining tube 810 may be rotated by twisting, rotating, or otherwise applying torque to the catheter 834. This allows a medical professional to transmit movement on a proximal portion of the catheter 834 to the restraining tube 810. After the spring coil 800 and restraining tube 810 are inserted into the tissue 830, the restraining tube 810 may be removed from about the spring coil 800. The spring coil 800 may be fixed relative to the restraining tube 810 for removal of the restraining tube 810 by abutting a pusher member 832 against a proximal end of the spring coil 810. The pusher member 832 may be more rigid than the restraining tube 810 such that the helical portion of the restraining tube 810 may substantially straighten out along the pusher member 832 as the restraining tube is removed proximally along the pusher member. Alternatively, the pusher member 832 may be helical in shape to accommodate the helical shape of the restraining tube 810.

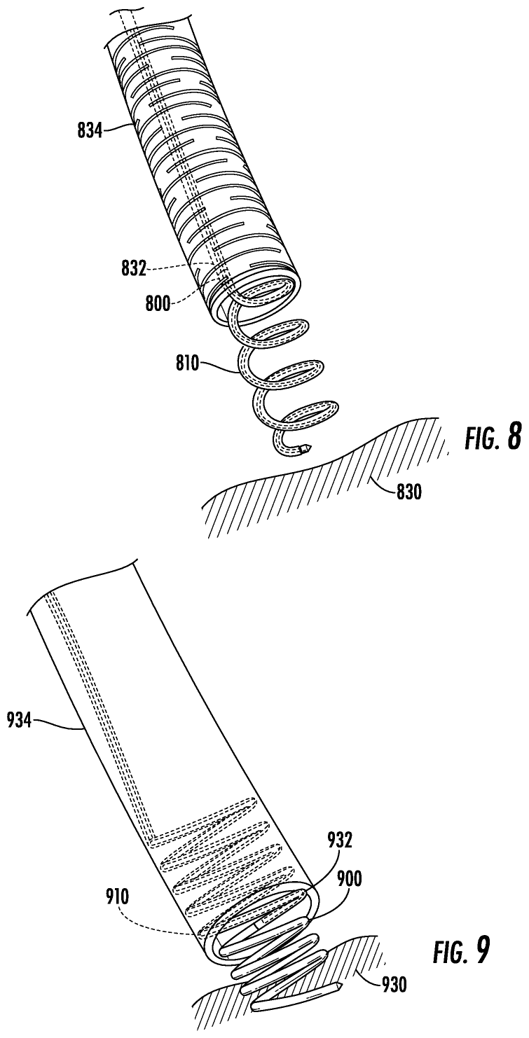

With reference to FIG. 9, an embodiment of a system for tightening, fastening, or anchoring in tissue according to the present disclosure is illustrated, which includes a hollow spring coil 900 with a restraining element 910 within the hollow spring coil 900 being delivered to a tissue 930 via a delivery catheter 934. The spring coil 900 and restraining element 910 may be inserted into the tissue 930 by rotating the coil 900 and element 910 into the tissue 930. This rotation may be performed by rotating the catheter 934 or by rotating the coil 900 and the tube 910 independent of the catheter 934. The restraining element 910 may be attached to the catheter 934 such that the restraining element 910 may be rotated by twisting, rotating, or otherwise applying torque to the catheter 934. This allows a medical professional to transmit movement on a proximal portion of the catheter 934 to the restraining element 910. The coil 900 and restraining element 910 have been inserted into the tissue 930, and the restraining element 910 is being removed. The hollow spring coil 900 may be fixed relative to the restraining element 910 for removal of the restraining element 910 by abutting a hollow pusher member 932, which may be coaxial with the restraining element 910 along the outside of the element 910, against a proximal end of the hollow spring coil 900. The pusher member 932 may be more rigid than the restraining element 910 such that the helical portion of the restraining element 910 may substantially straighten out along the pusher member 932. Alternatively, the pusher member 932 may be helical in shape to accommodate the helical shape of the restraining element 910 within the lumen of the pusher member 932.

In various embodiments, a proximal end of a restraining tube or a restraining element may be attached to a distal end of a delivery catheter. A retraining tube or restraining element may be attached via various adhesion methods such as, for example, welding, soldering, or brazing. A spring coil having a restraining tube or restraining element attached to a catheter may be delivered into a tissue by distally starting with a tip of the spring coil. Forward pressure may be applied to the catheter while simultaneously applying torque to turn and screw the system into the tissue. Once a desired position is reached, the catheter may be counter-rotated to remove the restraining tube or restraining element from the spring coil (with or without the assistance of a pusher member), leaving the spring coil in the tissue.

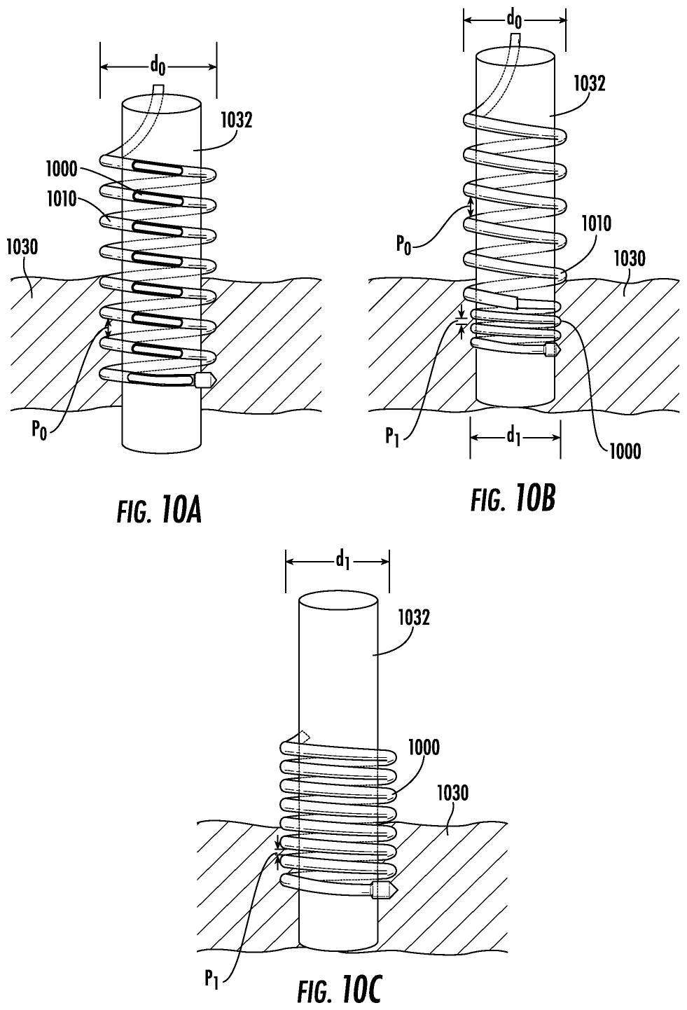

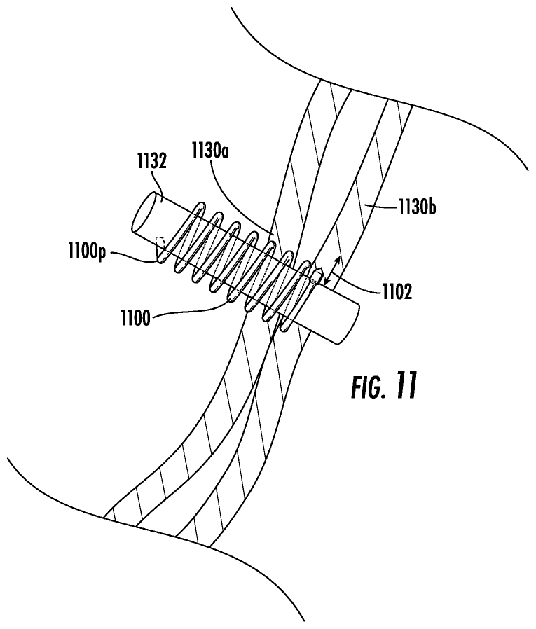

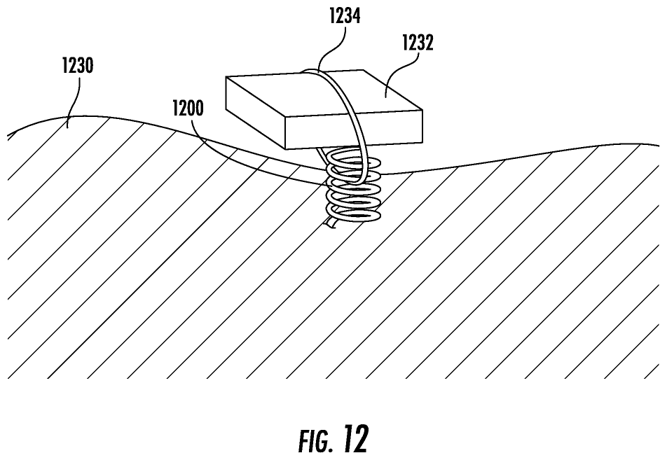

With reference to FIGS. 10A-10C, embodiments of a system for tightening, fastening, or anchoring in tissue according to the present disclosure are illustrated, which include a spring coil 1000 and a medical device 1032 being delivered into a tissue 1030. The spring coil 1000 is removably receivable within a restraining tube 1010, which are both secured about the medical device 1032 by holding the device 1032 with the restraining tube having a diameter d₀. The spring coil 1000 and restraining tube 1010 are inserted into the tissue 1032 as described herein, which may be along with a medical device 1032 that is held by the restraining tube 1010. The medical device 1032 may be inserted into one or more tissues 1030 into a desired position. Once the medical device 1032 is in the desired position, a spring coil 1000 and restraining tube 1010 may be deployed about the medical device 1032 to hold it in place. Alternatively, the spring coil 1000 and the restraining tube 1010 may be delivered with the medical device 1032 and may be integrated as part of the medical device 1032. As the coil 1000 and the restraining tube 1010 are inserted into the tissue 1030, the medical device 1032 is inserted through the tissue 1030, as illustrated in FIG. 10A. FIG. 10B illustrates the restraining tube 1010 being removed from the spring coil 1000. As the restraining tube 1010 is removed from the spring coil 1000, the spring coil 1000 transitions from a stressed configuration having a diameter and pitch of about the diameter d₀ and pitch p₀ of the restraining tube 1010 to an unstressed configuration having a smaller diameter d₁ and a smaller pitch p₁. The spring coil 1000 in the unstressed configuration having a smaller pitch p₁ and a smaller diameter d₁ pinches and squeezes the tissue 1030 and the medical device 1032. This pinching and squeezing substantially fixates the spring coil 1000 and medical device 1032 to the tissue 1030. FIG. 10C illustrates the spring coil 1000 and medical device 1032 in the delivered state within the tissue 1030 and with the restraining tube 1010 removed.

With reference to FIG. 11, an embodiment of a system for tightening, fastening, or anchoring in tissue according to the present disclosure is illustrated, which includes a medical device 1132 disposed through a first tissue 1130a and a second tissue 1130b, e.g., for draining from one body side to another. The medical device 1132 is fixated through the tissues 1130a, 1130b by the spring coil 1100. The spring coil 1100 extends through the first tissue 1030a but only extends into and not through the second tissue 1130b such that a tip 1102 of the coil 1100 is not exposed, but a proximal end 1100p of the coil 1100 is exposed for possible removal. The spring coil 1100 may also be a small enough coil to be fully or almost fully embedded in the two tissues 1130a, 1130b so neither end of the coil 1100 protrudes significantly from either tissue 1130a, 1130b. As the spring coil 1100 is screwed into second tissue 1130b and the spring coil 1100 transitions to an unstressed configuration as the restraining tube or element is removed, the second tissue 1130b is brought toward the first tissue 1130a, which may occur with or without the medical device 1132. The coil 1100 may also pinch and squeeze the tissue 1130a, 1130b adjacent the device to substantially fixate the device 1132, for example, as described and illustrated in relation to FIG. 10C.

With reference to FIG. 12, an embodiment of a system for tightening, fastening, or anchoring in tissue according to the present disclosure is illustrated, which includes a medical device 1232 secured to a spring coil 1200 delivered into a tissue 1230. The device 1232 is secured to the spring coil 1200, e.g., via a suture 1234 looped amongst a winding of the coil 1200 or a tethering mechanism such as an aperture, a loop, threads, or the like. Alternatively, a spring coil 1200 may be attached at or incorporated into a distal end of a device 1232 without needing a suture 1234, such as a cardiac defibrillator lead.

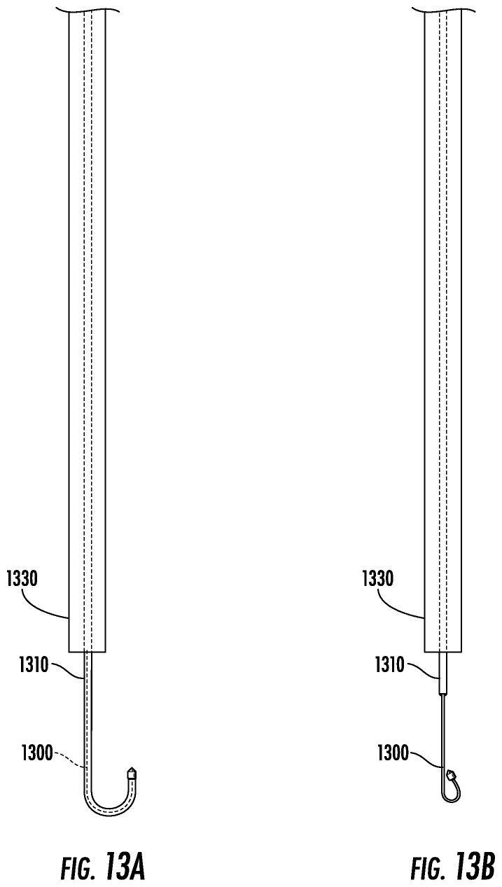

With reference to FIGS. 13A and 13B, an embodiment of a system for tightening, fastening, or anchoring in tissue according to the present disclosure is illustrated, which includes a spring coil 1300 within a restraining tube 1310. The system is being delivered via a catheter 1330. The spring coil 1300 is in the shape of a J-hook. The stressed spring coil 1300 within the restraining tube 1310 in FIG. 13A may be delivered into a tissue. In FIG. 13B, the restraining tube 1310 is moved proximally, revealing the spring coil 1300 in an unstressed configuration. The unstressed configuration of the spring coil 1300 has a tighter radius than that of the spring coil 1300 in the stressed configuration, assisting with anchoring the spring coil 1300 into tissue. In various embodiments, a spring coil may comprise a J-hook shape, a helical shape, or the like.

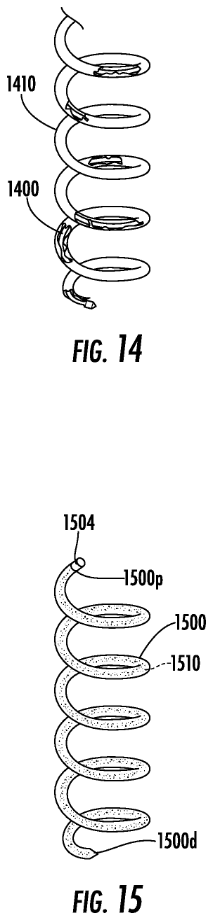

With reference to FIG. 14, an embodiment of a system for tightening, fastening, or anchoring in tissue according to the present disclosure is illustrated, which includes a spring coil 1410 in a stressed configuration within a rigid dissolvable restraining tube 1410. The coating may be dissolvable, biodegradable, bioerodable, bioresorbable, or the like. The coated coil 1400 may be delivered in the stressed configuration into a tissue. Over time, as the coating 1410 is exposed to biological fluids, the coating 1410 dissolves as shown in FIG. 14. As the coating 1410 substantially dissolves, resistive restraining forces keeping the spring coil in the stressed configuration dissipate with the coating 1410, permitting the coil 1400 to transition into the unstressed configuration and further anchor into the tissue.

With reference to FIG. 15, an embodiment of a system for tightening, fastening, or anchoring in tissue according to the present disclosure is illustrated, which includes a hollow spring coil 1500 containing a restraining element, which is a pressurized biocompatible fluid 1510. The pressurized fluid 1510 is kept under pressure because the distal end 1500d of the coil 1500 is closed and the proximal end 1500p is capped with a removable endcap 1504. The pressurized fluid 1510 forces the spring coil 1500 into the stressed configuration. The spring coil 1500 may be delivered in the stressed configuration with the fluid 1510 and endcap 1504. Once the coil 1500 is inserted into a tissue, the endcap 1504 may be removed, releasing the pressurized fluid 1510. As the hollow spring coil 1500 is depressurized of the fluid 1510, it transitions to the unstressed configuration and may further anchor into the tissue. Alternatively, the proximal end 1500p may be closed off without an endcap 1504. In this alternative embodiment, the spring coil 1500 may be punctured to release the pressurized fluid 1510.

In various embodiments of the present disclosure, one or more windings of a coil may have a diameter in a plane that is substantially parallel with the plane of one or more adjacent windings. These planes may be substantially transverse to a longitudinal axis of the coil or they may be angled with respect to the longitudinal axis, e.g., an angle less than about 90 degrees or an angle more than about 90 degrees. The angles of these planes and/or the pitch of adjacent coils may vary or may be constant throughout a length of the coil. The diameter of the windings may vary in size between a stressed configuration of the coil and an unstressed configuration of the coil. For example, the coil in a stressed configuration may have windings of a larger diameter than the windings in an unstressed configuration. As the coil transitions from the larger diameter windings in the stressed configuration to the smaller diameter windings in the unstressed configuration, the windings may squeeze the tissue and/or a medical device that is attached or within the coil.

In various embodiments, a pusher member may accommodate a restraining tube and/or fixate or push a spring coil. The pusher member may be substantially helical at a portion that accepts a restraining tube. The pusher member may be substantially straight and more rigid than the restraining tube such that the restraining tub substantially straightens out as it moves proximally along the pusher member. A pusher member may comprise polyimides, polyolefins, polyurethanes, any plastic with sufficient rigidity and flexibility, etc. A restraining tube, restraining element, and/or a spring coil may comprise steel, cobalt chromium alloys, nitinol, platinum, gold, palladium, polyimide, polyamide, polyurethane, combinations thereof, or the like. A material may be filled with radio-opaque fillers like barium and bismuth compounds, tungsten, etc. Exemplary materials for a spring coil may include steel, titanium, a polymer, a biocompatible material, an alloy, a combination thereof, or the like. A restraining tube and/or a pusher member may comprise the same or similar materials as the spring coil and may also comprise a polymer or the like. Non-medical embodiments may include device parts comprising a soft matrix, e.g., rubber.

In various embodiments, a method of tightening, fastening or anchoring in tissue may include inserting a catheter including the device into a patient. The device may include a spring. The spring may have a stressed configuration and an unstressed configuration. The device may include a restraint against the spring that may restrain the spring in the stressed configuration. The spring may be deployed from the catheter at least partially into a tissue. The restraint may be withdrawn from the spring such that at least a portion of the spring may transition from the stressed configuration to the unstressed configuration in the tissue to tighten, fasten or anchor in the tissue. The restraint may be a hollow tube about the spring. The restraint may be a coil within the spring. The spring may be deployed by rotating the catheter in a clockwise direction. The restraint may be withdrawn by rotating the catheter in a counter-clockwise direction. The restraint may be withdrawn by fixing a position of the spring relative to the restraint with a push member abutting a proximal end of the spring. The restraint may be attached to the distal end of the catheter. The device may be delivered into tissue about a secondary device, and removing the restraint allows the spring in the unstressed configuration to tighten the tissue about the secondary device. A secondary device may be positioned within the tissue. The spring may be deployed into the tissue about the secondary device. The restraint may be withdrawn such that the spring in the unstressed configuration may tighten the tissue about the secondary device.

In various embodiments, a portion (e.g., a proximal end, a distal end, a proximal tip, a distal tip, etc.) of a spring coil, restraining tube, or restraining element may include additional features such as an aperture or a loop configured to attached to a tether, a pusher, a snare, a basket, etc. for delivery, repositioning, or removal. A portion, such as a tip, may be rounded, polished, or otherwise treated. Such a tip may be substantially atraumatic. A portion may include a radiopaque material that may visually assist a medical professional with delivering, repositioning, or removing a device.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A system for tightening, fastening, or anchoring in tissue, comprising:
a spring coil having a distal end, a stressed configuration, and an unstressed configuration; and
a restraining tube having a lumen extending along a length of the tube, the spring coil removably receivable within the lumen to restrain the spring coil in the stressed configuration.

2. The system of claim 1, further comprising a tip with a distal tissue-penetrating portion on the distal end of the spring coil.

3. The system of claim 2, wherein a maximum diameter of the tip substantially matches and abuts an outer diameter of the restraining tube.

4. The system of claim 1, wherein the restraining tube is disposed over a proximal portion of the tip when the spring coil is in the stressed configuration.

5. The system of claim 1, wherein a plurality of windings of the spring coil have a stressed diameter in the stressed configuration and an unstressed diameter in the unstressed configuration, and wherein the unstressed diameter is smaller than the stressed diameter.

6. The system of claim 1, wherein the restraining tube is a dissolvable coating.

7. The system of claim 1, further comprising a delivery catheter having a distal end, wherein a proximal end of the restraining tube is fixedly attached to the distal end of the catheter.

8. The system of claim 7, wherein the spring coil is a helical coil and the restraining tube is a helical tube, and the catheter is rotatable to deliver the spring coil from within the lumen of the restraining tube.

9. The system of claim 8, wherein a diameter of the helical tube is greater than a diameter of the helical coil in the unstressed configuration, or windings of the helical coil have a pitch that is greater than windings of the helical coil in the unstressed configuration, or both.

10. A system for tightening, fastening or anchoring in tissue, comprising:
a hollow spring coil having a lumen extending along a length of the coil and having an open proximal end, a distal end, a stressed configuration, and an unstressed configuration; and
a restraining element disposed within the lumen, the restraining element removably receivable within the lumen to restrain the hollow spring coil in the stressed configuration.

11. The system of claim 10, further comprising a tip with a closed distal tissue-penetrating portion on the distal end of the hollow spring coil.

12. The system of claim 10, wherein a plurality of windings of the hollow spring coil have a stressed diameter in the stressed configuration and an unstressed diameter in the unstressed configuration, and wherein the unstressed diameter is smaller than the stressed diameter.

13. The system of claim 10, further comprising a delivery catheter having a distal end wherein a proximal end of the restraining element is attached to the distal end of the catheter.

14. The system of claim 10, wherein a diameter of the restraining element is greater than a diameter of the hollow spring coil in the unstressed configuration, or windings of the restraining element have a pitch that is greater than windings of the hollow spring coil in the unstressed configuration, or both.

15. A method of tightening, fastening or anchoring in tissue, comprising:
inserting a catheter including a device into a patient, the device comprising:
a spring having a stressed configuration and an unstressed configuration; and
a restraint against the spring that restrains the spring in the stressed configuration;
deploying the spring from the catheter at least partially into the tissue; and
withdrawing the restraint from the spring such that at least a portion of the spring transitions from the stressed configuration to the unstressed configuration in the tissue to tighten, fasten or anchor in the tissue.

16. The method of claim 15, wherein the restraint is a hollow tube about the spring.

17. The method of claim 15, wherein the restraint is a coil within the spring.

18. The method of claim 15, wherein deploying the spring further comprises rotating the catheter in a clockwise direction and withdrawing the restraint further comprises rotating the catheter in a counter-clockwise direction.

19. The method of claim 18, wherein withdrawing the restraint further comprises fixing a position of the spring relative to the restraint with a push member abutting a proximal end of the spring.

20. The method of claim 15, further comprising a secondary device positioned within the tissue, wherein the spring is deployed into the tissue about the secondary device, and wherein the restraint is withdrawn such that the spring in the unstressed configuration tightens the tissue about the secondary device.
